(19) United States
(12) Patent Application Publication
(10) Pub. No.: US 2022/0305061 A1
(43) Pub. Date: Sep. 29, 2022

(54) COMPOSITIONS DERIVED FROM HUMAN AMNION CELLS & RELATED METHODS

(71) Applicant: AXOLOTL BIOLOGIX, INC., Flagstaff, AZ (US)

(72) Inventors: Robert S. Kellar, Flagstaff, AZ (US); Robert B. Diller, Phoenix, AZ (US); Robert G. Audet, Dracut, MA (US); Eric T. Lee, Phoenix, AZ (US); Andrew T. Vo, Phoenix, AZ (US)

(73) Assignee: AXOLOTL BIOLOGIX, INC., Flagstaff, AZ (US)

(21) Appl. No.: 17/842,156

(22) Filed: Jun. 16, 2022

Related U.S. Application Data
(63) Continuation-in-part of application No. 17/290,662, filed on Apr. 30, 2021.
(60) Provisional application No. 63/211,062, filed on Jun. 16, 2021.
(60) Provisional application No. 62/895,444, filed on Sep. 3, 2019.
(60) Provisional application No. 62/884,987, filed on Aug. 9, 2019.

(51) Int. Cl. A61K 35/50; C12N 5/073; A61K 38/17; A61P 19/04; A61P 17/02; A61K 38/18; A61K 38/19; A61K 38/45; A61K 38/48; A61P 19/02

(52) U.S. Cl. CPC A61K 35/50; C12N 5/0605; A61K 38/1774; A61P 19/04; A61P 17/02; A61K 38/1875; A61K 38/177; A61K 38/1709; A61K 38/179; A61K 38/195; A61K 38/1841; A61K 38/193; A61K 38/45; C12Y 207/10001; C12Y 304/14005; A61K 38/4813; A61P 19/02

(57) ABSTRACT

A method for making an acellular human amnion-derived composition configured for therapeutic use is disclosed and generally includes the steps: obtaining amniotic membrane tissue; testing the amniotic membrane tissue for pathogens; washing the amniotic membrane tissue; manually removing blood-containing chorion tissue from the amniotic membrane tissue decellularizing the amniotic membrane tissue with xeno-free enzymes; collecting amniotic cells from the decellularized amniotic membrane tissue; seeding the amniotic cells for culture into xeno-free media formulated for mesenchymal stem cells; growing the amniotic cells to a specified confluency; collecting conditioned media; and freezing the collected conditioned media; wherein the method further includes irradiating the conditioned media.

### CROSS REFERENCE TO RELATED APPLICATIONS

This application claims benefit of U.S. Provisional Ser. No. 63/211,062 filed Jun. 16, 2021; the entire contents of which is hereby incorporated by reference.

This application is a continuation-in-part (CIP) of commonly owned U.S. Ser. No. 17/290,662, filed Apr. 30, 2021;

which is a National Stage application claiming benefit of International PCT/US20/45664 filed Aug. 10, 2020;

which claims benefit to both provisional application 62/895,444 filed Sep. 3, 2019 and to provisional application 62/884,987 filed Aug. 9, 2019;

the entire contents of each of which is hereby incorporated by reference.

### TECHNICAL FIELD

The invention relates to compositions derived from human amnion cells, and more particularly, growth-factor and cytokine-rich fluids derived from human amnion cells which are useful to treat a variety of ailments; and methods for making and using the same.

### BACKGROUND ART

There is currently no regenerative therapy that can be used to: (i) alleviate pain associated with connective tissue disease (CTD), and more particularly, degenerative joint disease, (ii) protect tissue from degenerative joint disease, and (iii) regenerate joint tissue to restore bio-function at the affected joint.

### SUMMARY OF INVENTION

### Technical Problem

Osteoarthritis is a degenerative joint disease, wherein cartilage wears away gradually causing pain, dysfunction and/or disability. While common in the hands and spine, osteoarthritis may also affect the hips, knees, feet, ankles, shoulders, and adjacent soft tissues.

Total joint replacement surgery is the gold standard treatment in patients with severe end-stage symptomatic osteoarthritis who have failed to respond to nonpharmacologic and pharmacologic management and who have significant impairment in their quality of life due to OA.

Pharmaceuticals that are often used to help relieve pain associated with osteoarthritis include: acetaminophen, nonsteroidal anti-inflammatory drugs (NSAIDs), and duloxetine (CYMBALTA®). While these pharmacotherapies may help to reduce pain, they are not regenerative and ultimately the patient may require total joint replacement surgery.

There is a need for a regenerative therapy that can be used to treat osteoarthritis. In particular, there is a need for a novel biologic treatment which can: (i) alleviate pain associated with osteoarthritis, (ii) protect tissue from degenerative joint disease, and (iii) regenerate joint tissue to restore bio-function at the affected joint.

While this disclosure may explicitly describe osteoarthritis, each of these problems spans other ailments associated more generally with connective tissue disease (CTD). As such, the scope of the invention may be applicable to other ailments associated with CTD, and some related diseases which do not involve connected tissue, such as, for example, hair follicle arrest and chronic skin wounds.

### Solution to Problem

Disclosed is a fluid composition configured for local injection at a site of connective tissue disease, and more particularly, in accordance with one embodiment, at a site of degenerative joint disease. The fluid composition comprises a growth factor and cytokine-rich fluid that is derived from human amnion cells, which we refer to herein as an “acellular human amnion-derived fluid composition” or “fluid”. The fluid comprises biomolecules that, when administered to a subject, especially at a local site of connective tissue disease, may induce: (i) tissue remodeling; (ii) cellular proliferation and differentiation; (iii) angiogenesis; (iv) cell migration; (v) anti-inflammatory responses; and (vi) anti-microbial activity.

### Advantageous Effects of Invention

Delivery by intra-articular or peri-articular injection can present the fluid, including cytokines and growth factors thereof, to the site of the connective tissue disease, such as a degenerative joint disease, which immediately and efficiently serves to provide therapeutic benefit at the location of interest.

In degenerative joint disease and chronic skin wounds, the anti-inflammatory biomolecules present in the fluid function to reduce inflammation, thereby helping to relieve pain.

Growth factors supporting epithelial proliferation and differentiation, angiogenesis, and remodeling are present in the fluid and function to repair and restore soft tissue.

Other features and benefits will be appreciated by one having skill in the art upon a thorough review of the instant disclosure.

### BRIEF DESCRIPTION OF DRAWINGS

FIG. 1 is a flow chart representing a method for making a growth-factor and cytokine-rich fluid derived from human amnion cells.

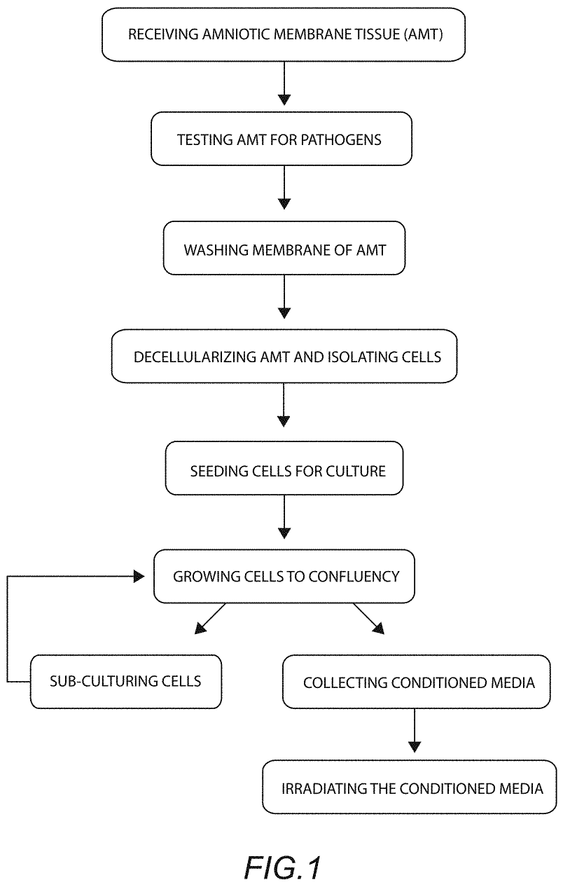

FIG. 2 shows a comparison of tissue-remodeling biomolecules as determined from bio-assays for each of CdM1 and CdM2.

FIG. 3 shows a comparison of proliferation and differentiation biomolecules as determined from bio-assays for each of CdM1 and CdM2.

FIG. 4 shows a comparison of angiogenic biomolecules as determined from bio-assays for each of CdM1 and CdM2.

FIG. 5 shows a comparison of cell migration biomolecules as determined from bio-assays for each of CdM1 and CdM2.

FIG. 6 shows a comparison of anti-inflammatory biomolecules as determined from bio-assays for each of CdM1 and CdM2.

FIG. 7 shows a comparison of other biomolecules, including anti-microbial, osteogenesis, pro-apoptotic, pro-inflammatory, and other uncategorized regenerative biomolecules, as determined from bio-assays for each of CdM1 and CdM2.

FIGS. 8(A-F) illustrate a clinical example of a non-healing wound (chronic skin wound), and subsequent treatment with an acellular human amnion-derived composition as-described herein.

FIGS. 9(A-B) illustrate a clinical example of a fibular fracture treated with an acellular human amnion-derived composition as-described herein.

FIGS. 10(A-B) illustrate a clinical example of degenerative joint disease, particularly ankle osteoarthritis, treated with an acellular human amnion-derived composition as-described herein.

FIGS. 11(A-D) illustrate the acellular human amnion-derived composition includes growth factors and cytokines produced by MSCs in culture.

### DESCRIPTION OF EMBODIMENTS

Disclosed herein is an acellular human amnion-derived fluid composition which has been surprisingly discovered to comprise a unique combination of biomolecules, such as growth factors and cytokines, which aid in the repair and restoration of soft tissue, especially in soft tissue affected by connective tissue disease, and more particularly, in soft tissue affected by degenerative joint disease.

For purposes herein, “connective tissue disease” means any disease that affects the parts of the body that connect the structures of the body together.

For purposes herein, “degenerative joint disease”, also referred to as “osteoarthritis”, means a type of arthritis that occurs when flexible tissue at the ends of bones wears down.

For purposes herein, “chronic skin wound” means any wound that does not heal in an orderly set of stages and in a predictable amount of time the way most wounds do; wounds that do not heal within three months are often considered chronic.

In a general embodiment, the invention is directed to a novel acellular human amnion-derived fluid composition, and methods for making and using the same.

In one aspect, an acellular human amnion-derived composition is disclosed, which comprises: one or more tissue-remodeling biomolecules; one or more proliferation biomolecules; one or more angiogenic biomolecules; one or more migration biomolecules; one or more anti-inflammatory biomolecules; and one or more anti-microbial biomolecules; wherein the composition is irradiated to achieve an ambient temperature stable acellular fluid.

For purposes herein, “tissue-remodeling biomolecules” means biomolecules that are implicated in the reorganization or renovation of existing tissues. The one or more tissue-remodeling biomolecules may comprise: cystatin B (CSTB); cystatin C (CST3); plasminogen activator inhibitor-1 (PAI-1); matrix metallopeptidase 1 (MMP1); matrix metallopeptidase 13 (MMP13); nidogen-1 (NID1); cathepsin L (CTSL); clusterin (CLU); extracellular matrix metalloproteinase inducer (EMMPRIN); TIMP metallopeptidase inhibitor 1 (TIMP1); TIMP metallopeptidase inhibitor 2 (TIMP2); decorin (DCN); transforming growth factor beta-induced protein ig-h3 (BIGH3); tumor necrosis factor superfamily member 11A (TNFRS11A); or a combination thereof.

For purposes herein, “proliferation biomolecules” means biomolecules that are implicated in the growth of new tissue. The one or more proliferation biomolecules may comprise: erb-b2 receptor tyrosine kinase 2 (ERBB2); dipeptidyl peptidase 4 (DPP4); epidermal growth factor receptor (EGFR); macrophage-colony stimulating factor (MCSF); activated leukocyte cell adhesion molecule (ALCAM); or a combination thereof.

For purposes herein, “angiogenic biomolecules” means biomolecules that are implicated in the formation of new blood vessels. The one or more angiogenic biomolecules may comprise: pentraxin 3 (PTX3); angiogenin (ANG); fms related tyrosine kinase 1 (FLT1); thrombospondin 1 (THBS1); urokinase-type plasminogen activator (uPA); transforming growth factor beta induced (TGFBI); angiopoietin 1 (ANG1); or a combination thereof.

For purposes herein, “migration biomolecules” means biomolecules that are implicated in the movement of cells to specific locations for tissue formation, wound healing and immune responses. The one or more migration biomolecules may comprise: syndecan 4 (SDC4); neuronal cell adhesion molecule (NRCAM); dickkopf WNT signaling pathway inhibitor 3 (DKK3); angiotensinogen (AGT); urokinase-type plasminogen activator receptor (UPAR); tyrosine protein kinase receptor UFO (AXL); or a combination thereof.

For purposes herein, “anti-inflammatory biomolecules” means biomolecules that are implicated in the reduction of inflammation. The one or more anti-inflammatory biomolecules may comprise: follistatin like 1 (FSTL1); galectin 1 (LGALS1); c-x-c motif chemokine 14 (CXCL14); or a combination thereof.

For purposes herein, “anti-microbial biomolecules” means biomolecules that are implicated in the killing of microorganisms or inhibition of their growth. The one or more anti-microbial biomolecules may comprise: beta-2-microglobulin (B2M).

For purposes herein, “osteogenesis biomolecules” means biomolecules that are implicated in the formation of bone. The composition may further comprise one or more osteogenesis biomolecules. The one or more osteogenesis biomolecules may comprise: follistatin like 3 (FSTL3); growth differentiation factor 15 (GDF15); or a combination thereof.

For purposes herein, “pro-inflammatory biomolecules” means biomolecules that are implicated in the promotion of inflammation and related inducement of an immune response. The composition may further comprise one or more pro-inflammatory biomolecules. The one or more pro-inflammatory biomolecules may comprise: tumor necrosis factor receptor 1 (TNFR1); single-chain type-1 glycoprotein (MIC2); galectin-3 (LGALS3); or a combination thereof.

For purposes herein, “pro-apoptotic biomolecules” means biomolecules that are implicated in promoting or causing apoptosis in cells. The composition may further comprise one or more pro-apoptotic biomolecules. The one or more pro-apoptotic biomolecules may comprise: Fas cell surface death receptor (FAS).

For purposes herein, “anti-proliferation biomolecules” means biomolecules that are implicated in the reduction of new tissue. The composition may further comprise one or more anti-proliferation biomolecules. The one or more anti-proliferation biomolecules may comprise: insulin-like growth factor-binding protein 4 (IGFBP4); ferritin (FTH1); or a combination thereof.

While certain examples of biomolecules are described, it should be understood that each biomolecule, while classified herein according to a particular function, may be alternatively classified as a different type of biomolecule where it has secondary function. For example, growth differentiation factor 15 (GDF15) is primarily an osteogenesis biomolecule; however, GDF15 has secondary function allowing it to be described as a tissue-remodeling biomolecule according to the knowledge and skill in the art.

In another aspect, a method for making an acellular human amnion-derived composition configured for therapeutic use is disclosed, the method comprises: obtaining amniotic membrane tissue; testing the amniotic membrane tissue for pathogens; washing the amniotic membrane tissue; manually removing blood-containing chorion tissue from the amniotic membrane tissue, decellularizing the amniotic membrane tissue with xeno-free enzymes; collecting cells from the decellularized amniotic membrane tissue; seeding the cells for culture into xeno-free media formulated for mesenchymal stem cells; growing the cells to a specified confluency; collecting conditioned media; and freezing the collected conditioned media; wherein the method further comprises: irradiating the frozen conditioned media.

The method may further comprise: freezing the collected conditioned media at −40° C. prior to irradiating the frozen conditioned media.

The method may further comprise: thawing the conditioned media; pooling one or more volumes of identical passages of the conditioned media from a common lot; aliquoting pooled conditioned media into desired volumes; and freezing the aliquots at −40° C.

The method may further comprise: subsequent to growing the cells to desired confluency, sub-culturing the cells and repeating the steps of: collecting conditioned media and irradiating the conditioned media obtained from the sub-cultured cells.

In yet another embodiment, a method for treating a subject suffering from connective tissue disease is disclosed, the method comprises: administering a therapeutically effective amount of an acellular human amnion-derived composition to soft tissue of the subject; whereby the subject is treated.

The method for treating a subject suffering from connective tissue disease is further distinguished wherein said acellular human amnion-derived composition comprises: one or more tissue-remodeling biomolecules; one or more proliferation biomolecules; one or more angiogenic biomolecules; one or more migration biomolecules; one or more anti-inflammatory biomolecules; and one or more anti-microbial biomolecules; wherein the composition is irradiated to render an acellular matrix. The connective tissue disease may comprise degenerative joint disease, other conditions which may benefit from the compositions and methods herein may include: hair follicle arrest and chronic skin wounds. Other connective tissue diseases, though not explicitly listed, may be similarly treated. In a preferred embodiment, the degenerative joint disease being treated comprises: ankle osteoarthritis.

The various embodiments of the invention will be better appreciated with the details as provided in the following examples with reference made to the related drawings.

### Example 1: Acellular Human Amnion-Derived Fluid Composition for Use in Soft Tissue Repair and Regeneration

### Tissue Preparation

Human placental tissue is obtained from a consenting donor in accordance with regulatory and other requirements. The tissue is placed in a sample container, and generally is suspended in natural fluid suspension. A sample is taken from the fluid suspension and tested for microbial contamination.

In a preferred embodiment, 1 mL of the fluid around the amnion is collected and tested for microbial contamination using 3M Petrifilms (https://www.3 m.com). The 3M Petrifilms can be used to test the cleanliness of surface samples or of a variety of samples in solution using known techniques.

Serology is performed on donor blood serum for screening purposes.

If serology or Petrifilms identify the presence of contamination, the membrane and all downstream cultures are destroyed.

### Cell Isolation & Membrane Decellularization

Pen-Strep antibiotic and mesenchymal stem cell culture media (culture media) were pre-warmed to room temperature. Three large sterile Erlenmeyer flasks were prepared for membrane washing with 200 mL of 1× Hank's Balanced Salt Solution (HBSS). The amniotic membrane was transferred aseptically into the first wash flask, closed with a sterile silicone stopper and placed on an orbital shaker for at least 20 minutes. After the first wash, the membrane was laid out on a sterile, stainless steel tray. With sterile gloves, the blood clots were manually rubbed or picked off of the membrane. Sections of the membrane where blood is trapped were untangled or excised until all visible blood clots were removed. Membrane was aseptically transferred to the second wash flask and 2 mL of 100× Pen-Strep antibiotic was added, the flask was placed on an orbital shaker for at least 20 minutes. During this incubation, three Erlenmeyer flasks were prepared for digestion, each with 100 mL of 1× TrypLE Select, 5 mM EDTA, and 1×HBSS.

The amnion was transferred to the first digestion flask for the preliminary TrypLE digest and incubated 10 minutes at 37° C., agitating the flask every 5 minutes. Following first digestion, the membrane was carefully moved to the second digestion flask, and incubated for 30 minutes at 37° C., agitating every 5 minutes. The first digest solution was properly disposed. After the second TrypLE digest was complete, the membrane was carefully moved to the third digestion flask and incubated for 30 minutes at 37° C., agitating every 5 minutes. The second TrypLE digest solution was properly disposed. After the third TrypLE digest was complete, 100 mL of 1×HBSS was added to the digest to dilute the TrypLE, and the flask was swirled to mix the solution. The membrane was carefully transferred to the third wash flask of 1×HBSS and the membrane swirled to dilute the TrypLE.

The solution from the third TrypLE digest was transferred into centrifuge tubes and centrifuged for 5 minutes at 200×g. The supernatant from each tube was carefully aspirated, leaving ˜0.5 mL supernatant above the pellet. The pellets were gently flicked to break them apart and triturated to resuspend them in the remaining supernatant. Resuspended cell pellets were pooled into a single 50 mL tube and 10 mL of cell culture media was added. The cell suspension was filtered through a 70-100 μm cell strainer into a fresh, sterile 50 ml tube. Cells were with a hemocytometer using Trypan blue to assess viability.

### Cell Seeding & Growth

Cells isolated from the amnion were triturated 10-20 times to produce a single cell suspension. Cells were seeded at approximately 10-30 million viable cells per T-25 flask. Additional culture media was added to the flasks, totaling 20 mL in a T25. One hundred microliters of 100× Pen-Strep was added to reach a final concentration of 0.5×. The flasks were incubated at 37° C. and 5% CO₂. Every 2-3 days, or as needed, each flask should be inspected on an inverted microscope for culture health and confluence. If the culture is less than 60% confluent, the flask is returned to the incubator and until it is 60-80% confluent. The flasks were subcultured and the media was collected. If a flask is determined to be over-seeded, then the density may be adjusted in accordance with known techniques.

### Media Collection

Conditioned media (CdM) is collected for cultures that are to be subcultured, at a target confluence of 60-80% or when the cells are not to be expanded/sub-cultured further at 80-100% confluence. The CdM is aseptically transferred from the cell culture flask into one or more 50 mL conical tubes. Using a pipette, 1 mL of the fluid product is withdrawn from each flask to test for microbial contamination. The 50 mL conical tube(s) of CdM are frozen for storage.

The cell culture flask(s) are appropriately disposed unless they will be used for sub-culturing.

### Cell Subculturing

When a cell culture flask reaches target confluence and is ready to be subcultured, the CdM is collected as described above. The flask is rinsed with 1×HBSS and trypsinized by adding 1× TrypLE Select solution at 1 mL/T25 flask or 2 mL/T75 flask and incubated for 5-15 minutes at 37° C., until 80% or more of the cells have rounded up and are still adherent. Cells are gently dislodged and digestion halted by adding 3 volumes of culture media and gently triturating the suspension down the flask wall several times to create a single cell suspension. Cell suspension is transferred to a conical tube and centrifuged 5 minutes at 200× g. Supernatant is removed, and tube is gently flicked to disrupt the pellet. We added 1-2 mL of culture media and triturated the cell suspension to create a single cell suspension. Cells are counted with a hemocytometer, using Trypan blue to assess viability. Flasks are seeded at 10,000-20,000 cells/cm² into suitable sized flasks to final volumes of 10-15 mL culture media/T25 and 20-30 mL/T75. Incubate at 37° C. and 5% CO₂.

### Packaging & Sterilization

Each 50 mL conical tube of thawed CdM which has passed Petrifilm testing is packaged for commercial distribution.

Using proper aseptic technique, the conditioned media from each conical tube is pipetted into the sterile cryovials. Each vial should receive the target volume of conditioned media with an additional 0.1 mL. Each conical tube should be pipetted into cryovials until there is only about 5 mL of conditioned media remaining, the remaining amount should be kept as a retention sample. After vials are filled, the corresponding caps should be securely tightened.

The vials are subsequently irradiated, between 5 kGy and 50 kGy, and more preferably between 14 kGy and 18 kGy using e-beam radiation, or as otherwise appreciated by one having skill in the art. Alternatively, the vials may be sterilized by gamma irradiation, X-Ray, and/or sterile filtration. Sterility may be assessed by sterilization validation or by 14-day culture.

### Administration and Delivery

In preparation for use, a vial containing the fluid composition is optionally thawed (if frozen) and loaded in a syringe. Alternatively, the preparation may be provided in a pre-filled syringe. A physician administers the fluid composition by intra-articular or peri-articular injection at the site of degenerative joint disease. For chronic wounds, the fluid composition is injected within the wound bed and into the wound margins.

In some embodiments, the preparation is manufactured into a topical formulation as would be appreciated by one having skill in the art. The topical formulation may be applied to the skin of a patient.

### Example 2: Characterization of Amnion-Derived Fluid Composition

A first conditioned media (“CdM1”) was obtained in accordance with the methods set forth in Example 1, above, and making use of human placental tissue from a first consenting donor. Note that “conditioned media” as used herein refers to the acellular human amnion-derived composition, which terms are interchangeable for purposes of this disclosure.

As a control, the same process was performed as set forth in Example 1, above, in absence of human placental tissue, which we refer to as a “first control” for reason that it was produced in concert with the first conditioned media. Biomolecules within the resulting first conditioned media were screened using conventional bio-assays, such as cellular proliferation assays. Comparison of select biomolecules detected between the first conditioned media and first control were quantified as percent (%) above control.

Similarly, a second conditioned media (“CdM2”) was obtained in accordance with the methods set forth in Example 1, above, and making use of human placental tissue from a second consenting donor.

As a control, the same process was performed as set forth in Example 1, above, in absence of human placental tissue, which we refer to as a “second control” for reason that it was produced in concert with the second conditioned media. Biomolecules within the resulting second conditioned media were screened using conventional bio-assays. Comparison of select biomolecules detected between the second conditioned media and second control were quantified as percent (%) above control.

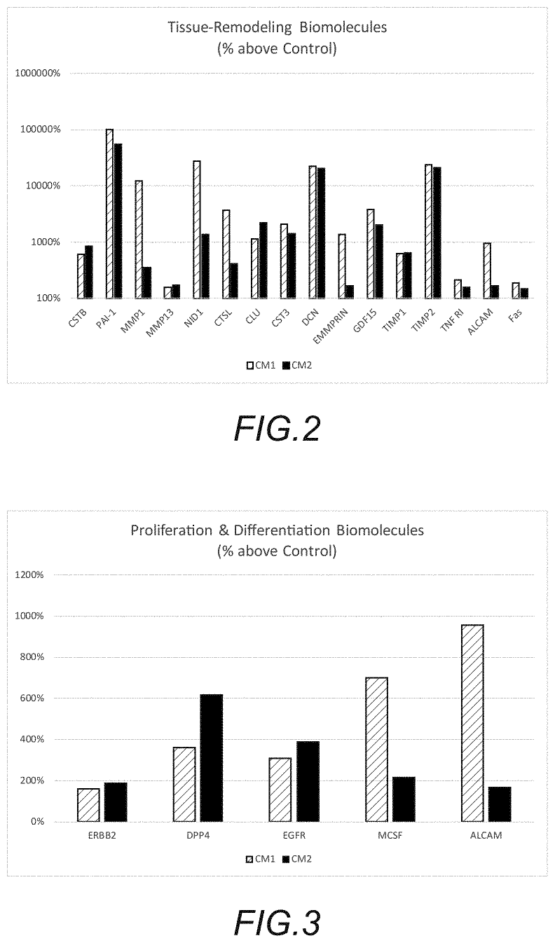

FIG. 2 shows a comparison of tissue-remodeling biomolecules as determined from bio-assays for each of CdM1 and CdM2. The tissue-remodeling biomolecules include: cystatin B (CSTB); cystatin C (CST3); plasminogen activator inhibitor-1 (PAI-1); matrix metallopeptidase 1 (MMP1); matrix metallopeptidase 13 (MMP13); nidogen-1 (NID1); cathepsin L (CTSL); clusterin (CLU); extracellular matrix metalloproteinase inducer (EMMPRIN); TIMP metallopeptidase inhibitor 1 (TIMP1); TIMP metallopeptidase inhibitor 2 (TIMP2); decorin (DCN); and growth differentiation factor 15 (GDF15). One or more of these biomolecules can be isolated or extracted from the fluid using column chromatography or other known techniques; thus, one or more of these biomolecules, and up to each of these biomolecules, may be provided in a select embodiment of the invention. We note both CdM1 and CdM2 produced these tissue-remodeling biomolecules at significant percent above control, suggesting that our method as outlined in Example 1, above, when practiced with human placental tissue, produces desirable biomolecules associated with tissue-remodeling.

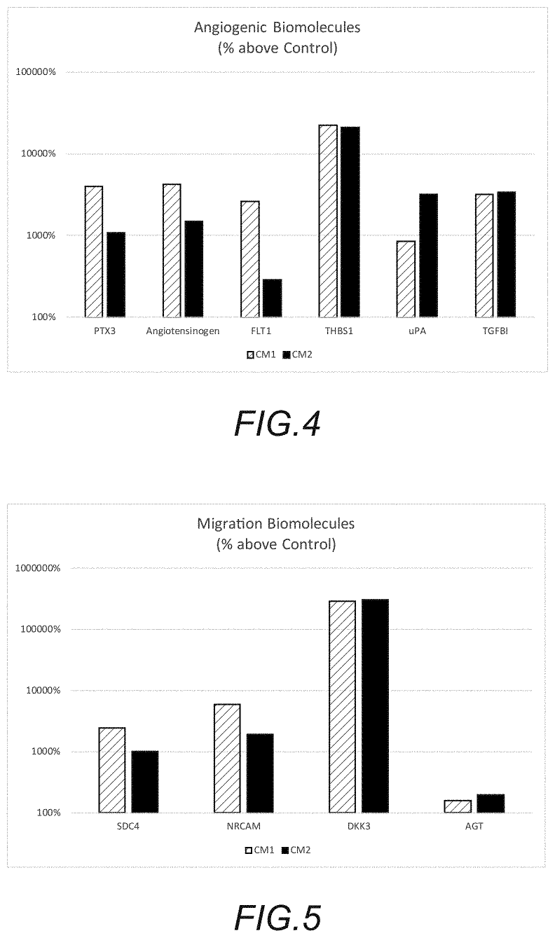

FIG. 3 shows a comparison of proliferation and differentiation biomolecules as determined from bio-assays for each of CdM1 and CdM2. The proliferation and differentiation biomolecules include: erb-b2 receptor tyrosine kinase 2 (ERBB2); dipeptidyl peptidase 4 (DPP4); epidermal growth factor receptor (EGFR); macrophage-colony stimulating factor (MCSF); and activated leukocyte cell adhesion molecule (ALCAM). One or more of these biomolecules can be isolated or extracted from the fluid using column chromatography or other known techniques; thus, one or more of these biomolecules, and up to each of these biomolecules, may be provided in a select embodiment of the invention. We note both CdM1 and CdM2 produced these proliferation and differentiation biomolecules at significant percent above control, suggesting that our method as outlined in Example 1, above, when practiced with human placental tissue, produces desirable biomolecules associated with proliferation and differentiation.

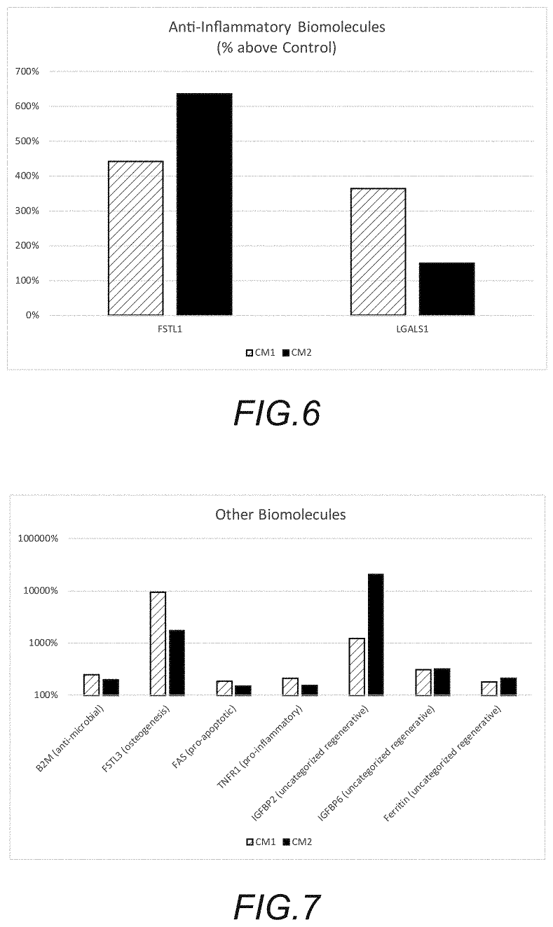

FIG. 4 shows a comparison of angiogenic biomolecules as determined from bio-assays for each of CdM1 and CdM2. The angiogenic biomolecules include: pentraxin 3 (PTX3); angiogenin (ANG); fms related tyrosine kinase 1 (FLT1); thrombospondin 1 (THBS1); urokinase-type plasminogen activator (uPA); and transforming growth factor beta induced (TGFBI). One or more of these biomolecules can be isolated or extracted from the fluid using column chromatography or other known techniques; thus, one or more of these biomolecules, and up to each of these biomolecules, may be provided in a select embodiment of the invention. We note both CdM1 and CdM2 produced these angiogenic biomolecules at significant percent above control, suggesting that our method as outlined in Example 1, above, when practiced with human placental tissue, produces desirable biomolecules associated with angiogenesis (blood vessel formation).

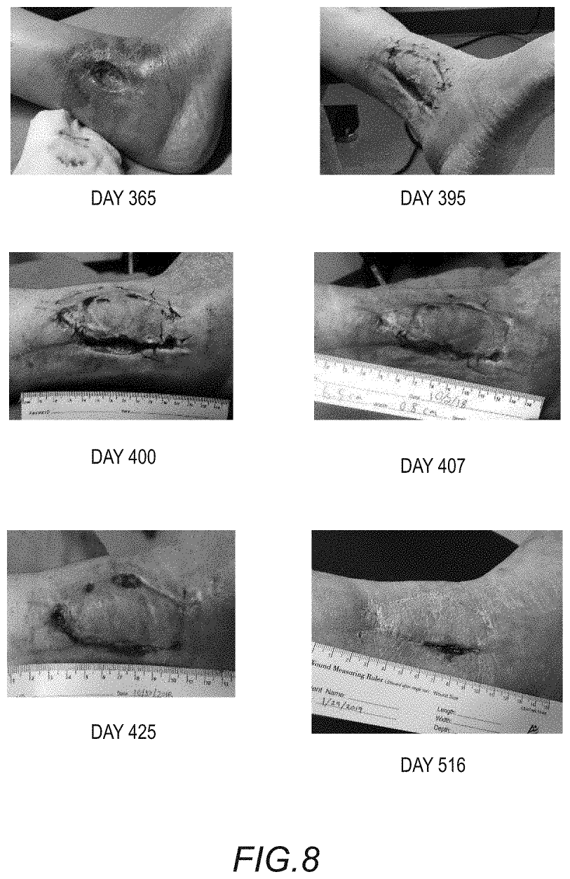

FIG. 5 shows a comparison of cell migration biomolecules as determined from bio-assays for each of CdM1 and CdM2. The cell migration biomolecules include: syndecan 4 (SDC4); neuronal cell adhesion molecule (NRCAM); dickkopf WNT signaling pathway inhibitor 3 (DKK3); and angiotensinogen (AGT). One or more of these biomolecules can be isolated or extracted from the fluid using column chromatography or other known techniques; thus, one or more of these biomolecules, and up to each of these biomolecules, may be provided in a select embodiment of the invention. We note both CdM1 and CdM2 produced these cell migration biomolecules at significant percent above control, suggesting that our method as outlined in Example 1, above, when practiced with human placental tissue, produces desirable biomolecules associated with cell migration.

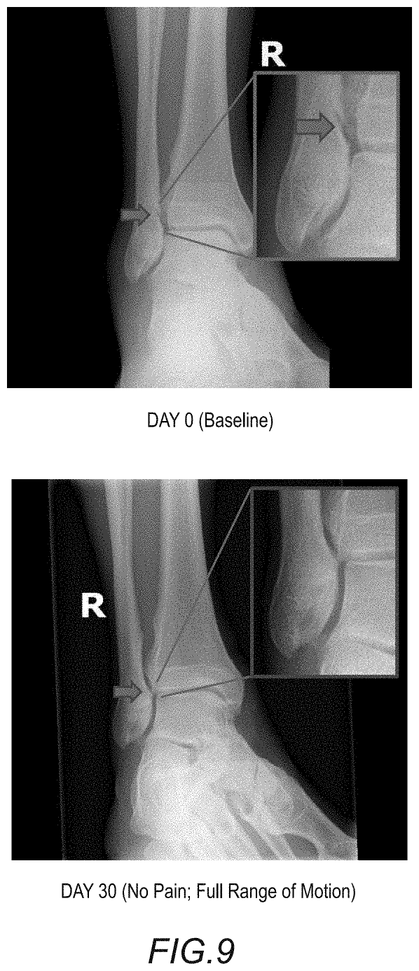

FIG. 6 shows a comparison of anti-inflammatory biomolecules as determined from bio-assays for each of CdM1 and CdM2. The anti-inflammatory biomolecules include: follistatin like 1 (FSTL1); and galectin 1 (LGALS1). One or more of these biomolecules can be isolated or extracted from the fluid using column chromatography or other known techniques; thus, one or more of these biomolecules, and up to each of these biomolecules, may be provided in a select embodiment of the invention. We note both CdM1 and CdM2 produced these anti-inflammatory biomolecules at significant percent above control, suggesting that our method as outlined in Example 1, above, when practiced with human placental tissue, produces desirable biomolecules associated with anti-inflammatory activity.

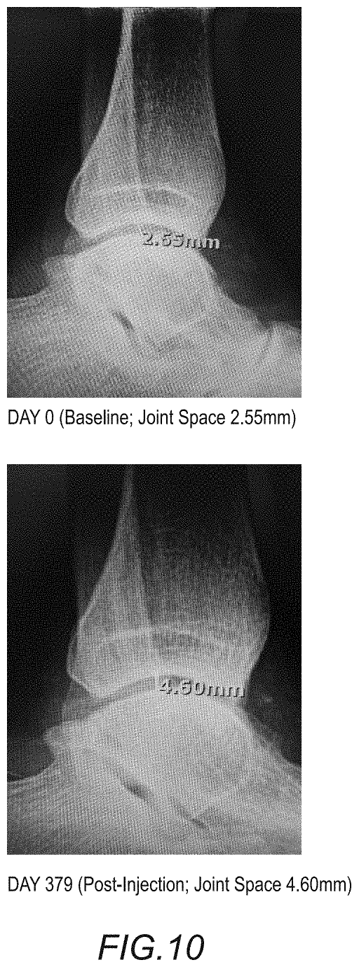

FIG. 7 shows a comparison of other biomolecules, including anti-microbial, osteogenesis, pro-apoptotic, pro-inflammatory, and other uncategorized regenerative biomolecules, as determined from bio-assays for each of CdM1 and CdM2. These biomolecules include: beta-2-microglobulin (B2M) which is an anti-microbial biomolecule; follistatin like 3 (FSTL3) which is an osteogenesis biomolecule; Fas cell surface death receptor (FAS) which is a pro-apoptotic biomolecule; tumor necrosis factor receptor 1 (TNFR1) which is a pro-inflammatory biomolecule; and other uncategorized regenerative biomolecules including: IGFBP2, IGFBP6, and Ferritin. One or more of these biomolecules can be isolated or extracted from the fluid using column chromatography or other known techniques; thus, one or more of these biomolecules, and up to each of these biomolecules, may be provided in a select embodiment of the invention. We note both CdM1 and CdM2 produced each of these biomolecules at significant percent above control, suggesting that our method as outlined in Example 1, above, when practiced with human placental tissue, produces desirable anti-microbial, osteogenesis, pro-apoptotic, pro-inflammatory, and other uncategorized regenerative biomolecules.

### Example 3: Alternative Characterization of Amnion-Derived Fluid Composition

A Bradford assay was conducted on various manufacturing passages of the conditioned media and controls (“Prime”) that are in absence of the human placental tissue. The manufacturing passages were manufactured at different points in time, at different facilities, with different donor placentas. In total, there were four manufacturing passages where each passage had one control and two to four lots.

Various growth factors from the conditioned media were measured and compared with its corresponding control. Table 1 below shows a list of growth factors that presented average concentrations in the manufacturing passages were significantly higher than the average concentrations in the control.

TABLE 1
Growth Factor Concentrations (pg/ml)

| | Control | Conditioned | Percent |
|---|---|---|---|
| Gene ID | Mean | Media Mean | Increase (%) |
| ANG | 1.62 | 95.62 | 5802.5 |
| ANG1 | 30.01 | 1022.59 | 3307.5 |
| AXL | 8.94 | 32.73 | 266.1 |
| B2M | 88.29 | 234.56 | 165.7 |
| BIGH3 | LOD | 1608.38 | NA |
| CLCX14 | LOD | 30.47 | NA |
| CLU | 170.11 | 2899.56 | 1604.5 |
| CST3 | 54.51 | 1531.36 | 2709.3 |
| CSTB | 31.62 | 249.81 | 690.0 |
| CSTL | 10.25 | 524.46 | 5016.7 |
| DCN | 29.56 | 1186.64 | 3914.3 |
| DKK3 | LOD | 2455.53 | NA |
| DPPIV | 26.33 | 171.6 | 551.7 |
| EMMPRIN | 16.98 | 126.13 | 642.8 |
| FAS | 5.13 | 12.55 | 144.6 |
| FLT1 | 61.79 | 636.74 | 930.5 |
| FSTL1 | 603.51 | 3159.91 | 423.6 |
| FTH1 | 1423.44 | 3412.05 | 139.7 |
| GDF15 | 0.27 | 17.88 | 6522.2 |
| IGFBP4 | 1063.25 | 29541.2 | 2678.4 |
| LDLR | LOD | 79.57 | NA |
| LGALS1 | 293.52 | 2111.98 | 619.5 |
| LGALS3 | 3.67 | 20.99 | 471.9 |
| MCSF | 0.71 | 15.13 | 2031.0 |
| MET | LOD | 14.47 | NA |
| MIC2 | 157.75 | 465.42 | 195.0 |
| MMP1 | 15.76 | 506.04 | 3110.9 |
| NID | 22.98 | 5314.69 | 23027.5 |
| PAI1 | 32.16 | 3615.37 | 11141.8 |
| PTX 3 | LOD | 2981.04 | NA |
| SDC4 | 0.05 | 2.56 | 5020.0 |
| TGFB1 | 3.99 | 128.28 | 3115.0 |
| TIMP1 | 168.44 | 2104.31 | 1149.3 |
| TIMP2 | 20.48 | 3706.9 | 18000.1 |
| TNFRSF11A | 20.01 | 1195.73 | 5875.7 |
| TSP1 | 84.56 | 22572.4 | 26593.9 |
| UPAR | 40.63 | 384.4 | 846.1 |

In one embodiment, an acellular human amnion-derived composition is disclosed. The composition comprises a plurality of biomolecules including tissue-remodeling biomolecules, proliferation biomolecules, angiogenic biomolecules, migration biomolecules, anti-inflammatory biomolecules, anti-microbial biomolecules, osteogenesis biomolecules, pro-apoptotic biomolecules, pro-inflammatory biomolecules, anti-proliferation biomolecules, or a combination thereof.

The tissue-remodeling biomolecules include: cystatin B (CSTB); cystatin C (CST3); plasminogen activator inhibitor-1 (PAI-1); matrix metallopeptidase 1 (MMP1); nidogen-1 (NID1); cathepsin L (CTSL); clusterin (CLU); extracellular matrix metalloproteinase inducer (EMMPRIN); TIMP metallopeptidase inhibitor 1 (TIMP1); TIMP metallopeptidase inhibitor 2 (TMP2); decorin (DCN); tumor necrosis factor superfamily member 11A (TNFRS11A); transforming growth factor beta-induced protein ig-h3 (BIGH3). One or more of these biomolecules can be isolated or extracted from the fluid using column chromatography or other known techniques; thus, one or more of these biomolecules, and up to each of these biomolecules, may be provided in a select embodiment of the invention. Table 1 shows that the average of the conditioned media produced these tissue-remodeling biomolecules at significant percent above control, suggesting that the method outlined in Example 1, above, when practiced with human placental tissue, produces desirable biomolecules associated with tissue-remodeling. In some embodiments, the composition comprises the transforming growth factor beta-induced protein ig-h3 (BIGH3), which was not detected in the control and showed significant quantities in the manufacturing passages.

The proliferation and differentiation biomolecules include: dipeptidyl peptidase 4 (DPP4); macrophage-colony stimulating factor (MCSF). One or more of these biomolecules can be isolated or extracted from the fluid using column chromatography or other known techniques; thus, one or more of these biomolecules, and up to each of these biomolecules, may be provided in a select embodiment of the invention. Table 1 shows that the average of the conditioned media produced these proliferation and differentiation biomolecules at significant percent above control, suggesting that the method as outlined in Example 1, above, when practiced with human placental tissue, produces desirable biomolecules associated with proliferation and differentiation.

The angiogenic biomolecules include: pentraxin 3 (PTX3); angiogenin (ANG); fms related tyrosine kinase 1 (FLT1); thrombospondin 1 (THBS1); transforming growth factor beta induced (TGFBI); angiopoietin 1 (ANG1). One or more of these biomolecules can be isolated or extracted from the fluid using column chromatography or other known techniques; thus, one or more of these biomolecules, and up to each of these biomolecules, may be provided in a select embodiment of the invention. Table 1 shows that the average of the conditioned media produced these angiogenic biomolecules at significant percent above control, suggesting that the method as outlined in Example 1, above, when practiced with human placental tissue, produces desirable biomolecules associated with angiogenesis (blood vessel formation). In some embodiments, the composition comprises pentraxin 3 (PTX3), which was not detected in the control and showed significant quantities in the manufacturing passages.

The cell migration biomolecules include: syndecan 4 (SDC4); dickkopf WNT signaling pathway inhibitor 3 (DKK3); tyrosine protein kinase receptor UFO (AXL); urokinase-type plasminogen activator receptor (UPAR). One or more of these biomolecules can be isolated or extracted from the fluid using column chromatography or other known techniques; thus, one or more of these biomolecules, and up to each of these biomolecules, may be provided in a select embodiment of the invention. Table 1 shows that the average of the conditioned media produced these cell migration biomolecules at significant percent above control, suggesting that the method as outlined in Example 1, above, when practiced with human placental tissue, produces desirable biomolecules associated with cell migration. In some embodiments, the composition comprises dickkopf WNT signaling pathway inhibitor 3 (DKK3), which was not detected in the control and showed significant quantities in the manufacturing passages.

The anti-inflammatory biomolecules include: follistatin like 1 (FSTL1); galectin 1 (LGALS1); c-x-c motif chemokine 14 (CXCL14). One or more of these biomolecules can be isolated or extracted from the fluid using column chromatography or other known techniques; thus, one or more of these biomolecules, and up to each of these biomolecules, may be provided in a select embodiment of the invention. Table 1 shows that the average of the conditioned media produced these anti-inflammatory biomolecules at significant percent above control, suggesting that the method as outlined in Example 1, above, when practiced with human placental tissue, produces desirable biomolecules associated with anti-inflammatory activity. In some embodiments, the composition comprises c-x-c motif chemokine 14 (CXCL14), which was not detected in the control and showed significant quantities in the manufacturing passages.

The pro-inflammatory biomolecules include: galectin-3 (LGALS3); HGF receptor (MET); single-chain type-1 glycoprotein (MIC2). One or more of these biomolecules can be isolated or extracted from the fluid using column chromatography or other known techniques; thus, one or more of these biomolecules, and up to each of these biomolecules, may be provided in a select embodiment of the invention. Table 1 shows that the average of the conditioned media produced these pro-inflammatory biomolecules at significant percent above control, suggesting that the method as outlined in Example 1, above, when practiced with human placental tissue, produces desirable biomolecules associated with pro-inflammatory activity. In some embodiments, the composition comprises HGF receptor (MET), which was not detected in the control and showed significant quantities in the manufacturing passages.

The anti-proliferation biomolecules include: insulin-like growth factor-binding protein 4 (IGFBP4); ferritin (FTH1). One or more of these biomolecules can be isolated or extracted from the fluid using column chromatography or other known techniques; thus, one or more of these biomolecules, and up to each of these biomolecules, may be provided in a select embodiment of the invention. Table 1 shows that the average of the conditioned media produced these anti-proliferation biomolecules at significant percent above control, suggesting that the method as outlined in Example 1, above, when practiced with human placental tissue, produces desirable biomolecules associated with anti-proliferation activity.

Other biomolecules, including anti-microbial, osteogenesis, pro-apoptotic, and other uncategorized regenerative biomolecules, were detected to show substantial growth between the control and the conditioned media. These biomolecules include: beta-2-microglobulin (B2M) which is an anti-microbial biomolecule; growth differentiation factor-15 (GDF15) which is an osteogenesis biomolecule; tumor necrosis factor receptor superfamily member 6 (FAS) which is a pro-apoptotic biomolecule; and other uncategorized regenerative biomolecules including: low density lipoprotein receptor (LDLR) which is known for cell metabolism, capillary regression, and anti-angiogenesis. One or more of these biomolecules can be isolated or extracted from the fluid using column chromatography or other known techniques; thus, one or more of these biomolecules, and up to each of these biomolecules, may be provided in a select embodiment of the invention. Table 1 shows that the average of the conditioned media produced each of these biomolecules at significant percent above control, suggesting that the method as outlined in Example 1, above, when practiced with human placental tissue, produces desirable anti-microbial, osteogenesis, pro-apoptotic, and other uncategorized regenerative biomolecules.

The acellular human amnion-derived composition can be used in treating a subject suffering from a connective tissue disease, hair follicle arrest, or a chronic skin wound.

### Example 4: Acellular Human Amnion-Derived Composition for Chronic Wounds

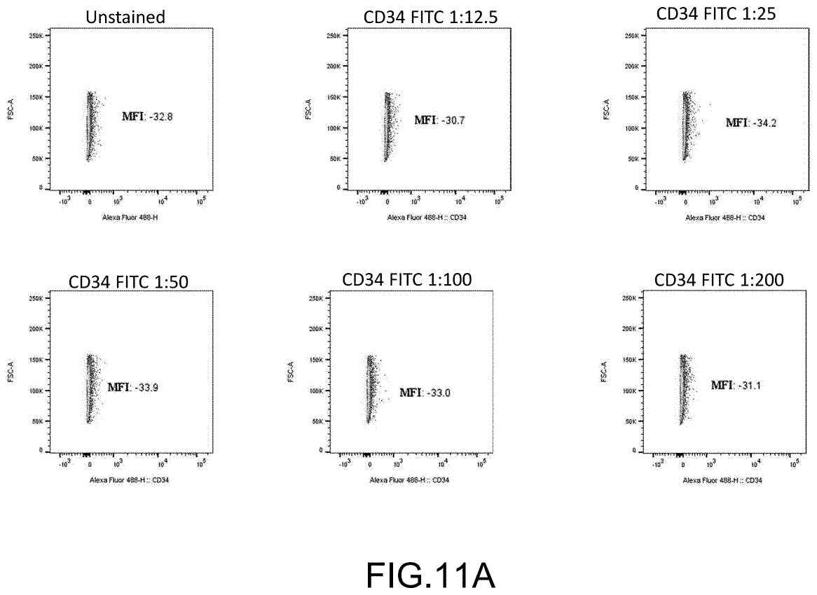

FIG. 8 depicts a clinical example of a forty-seven-year-old patient with broken ankle. Nearly one year after the injury (Day 365), the surgical site remained open. An acellular human amnion-derived composition, as described herein, was applied at various intervals (Day 365, Day 395, Day 400, Day 407, Day 425, and Day 516, respectively) and photographic data obtained. Within five months the wound was substantially healed. This example shows the clinical utility associated with the acellular human amnion-derived composition with respect to applications related to wound healing.

### Example 5: Acellular Human Amnion-Derived Composition for Fibular Fracture

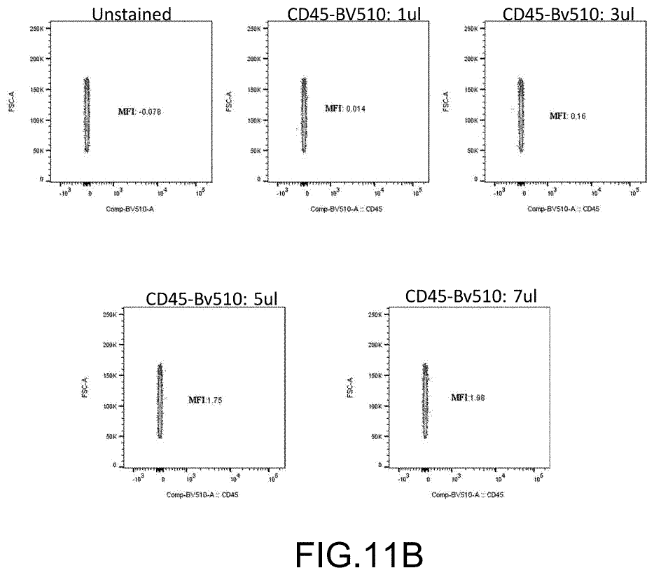

FIG. 9 depicts images obtained from a thirty-nine-year-old patient with a fibular fracture. A first image taken at Day 0 (baseline) shows the initial state of the fibular fracture. At this time, the patient complained of pain and restricted motion. The patient was treated with an acellular human amnion-derived composition as described herein. After thirty days (Day 30), the patient indicated the pain completely subsided and further demonstrated a full range of motion. An image was obtained, and the fracture is visibly healed.

### Example 6: Acellular Human Amnion-Derived Composition for Ankle Osteoarthritis

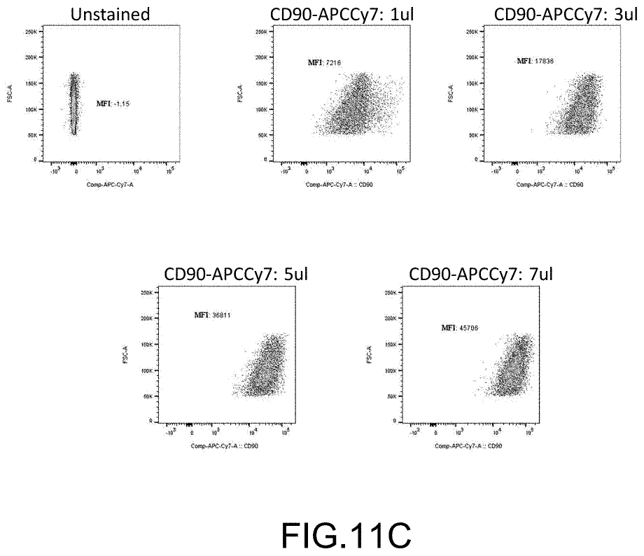

FIG. 10 depicts images obtained from a patient with ankle osteoarthritis. Pre-injection, a weight-bearing ankle x-ray image was obtained (Day 0; baseline), which revealed a joint space of about 2.55 mm. Regenerative therapy was achieved by later administering an acellular human amnion-derived composition as described herein. After about one year from the injury baseline (Day 379) another x-ray image was obtained post-injection, which reveals soft tissue regeneration in the joint space, which at that time measured 4.60 mm (just over 2.0 mm or about 80% improvement.

### Example 7: Mesenchymal Stromal Cells Expressing CD90 and CD105 After Multiple Passages

In certain embodiments, it is preferred to utilize mesenchymal stromal cells (MSCs) for up to four passages, or less. The reason for limiting use of MSCs to four passages is to ensure an optimal yield of growth factors and cytokines in the resulting acellular human amnion-derived composition.

Mesenchymal stromal cells are multipotent progenitor cells used in several cell therapies. MSCs are characterized by the expression of CD73, CD90, and CD105 cell markers, and the absence of CD34, CD45, CD11a, CD19, and HLA-DR cell markers. CD90 is a glycoprotein present in the MSC membranes and also in adult cells and cancer stem cells.

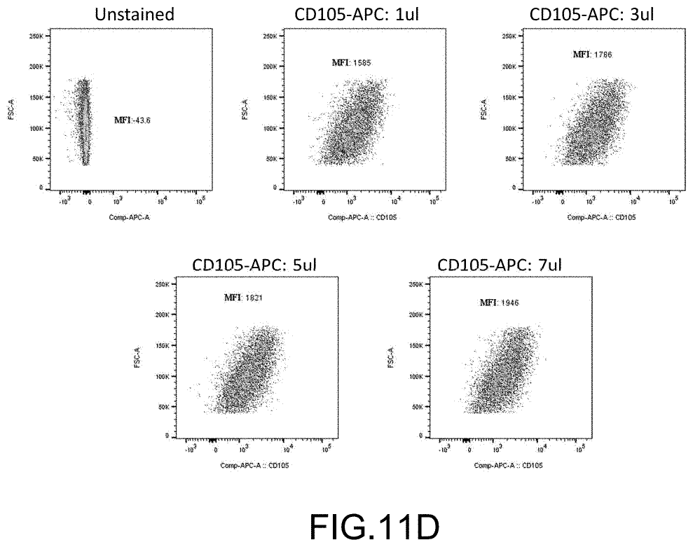

In this example, cells were tested after five (5) passages to identify expression of CD34; CD45; CD90; and CD105. Results of a standard bio assay reveal the MSCs produced according to the process described herein do not express CD34 and CD45, but do express CD90 and CD105. See FIGS. 11(A-D). Accordingly, the human amnion-derived composition includes cytokines and growth factors derived from MSCs.

### INDUSTRIAL APPLICABILITY

The invention is applicable to the medical industry as it encompasses a biologic conditioned media, namely, an acellular human amnion-derived composition, which is useful as a therapeutic for soft tissue repair and remodeling, especially that which is desired in response to connective tissue disease, and more particularly, to osteoarthritis.

What is claimed is:

1. An acellular human amnion-derived composition, comprising:
one or more tissue-remodeling biomolecules selected from the group consisting of: cystatin B (CSTB); cystatin C (CST3); plasminogen activator inhibitor-1 (PAI-1); matrix metallopeptidase 1 (MMP1); nidogen-1 (NID1); cathepsin L (CTSL); clusterin (CLU); extracellular matrix metalloproteinase inducer (EMMPRIN); TIMP metallopeptidase inhibitor 1 (TIMP1); TIMP metallopeptidase inhibitor 2 (TIMP2); decorin (DCN); tumor necrosis factor superfamily member 11A (TNFRS11A); transforming growth factor beta-induced protein ig-h3 (BIGH3); or a combination thereof,
one or more proliferation biomolecules selected from the group consisting of: dipeptidyl peptidase 4 (DPP4); macrophage-colony stimulating factor (MCSF); or a combination thereof,
one or more angiogenic biomolecules selected from the group consisting of: pentraxin 3 (PTX3); angiogenin (ANG); fms related tyrosine kinase 1 (FLT1); thrombospondin 1 (THBS1); transforming growth factor beta induced (TGFBI); angiopoietin 1 (ANG1); or a combination thereof,
one or more migration biomolecules selected from the group consisting of: syndecan 4 (SDC4); dickkopf WNT signaling pathway inhibitor 3 (DKK3); tyrosine protein kinase receptor UFO (AXL); urokinase-type plasminogen activator receptor (UPAR); or a combination thereof,
one or more anti-inflammatory biomolecules selected from the group consisting of: follistatin like 1 (FSTL1); galectin 1 (LGALS1); c-x-c motif chemokine 14 (CXCL14); or a combination thereof, and
one or more anti-microbial biomolecules including beta-2-microglobulin (B2M),
for use in treating a subject suffering from a connective tissue disease, hair follicle arrest, or a chronic skin wound.

2. The acellular human amnion-derived composition of claim 1, further comprising one or more osteogenesis biomolecules including growth differentiation factor-15 (GDF15).

3. The acellular human amnion-derived composition of claim 1, further comprising one or more pro-apoptotic biomolecules including tumor necrosis factor receptor superfamily member 6 (FAS).

4. The acellular human amnion-derived composition of claim 1, further comprising one or more pro-inflammatory biomolecules selected from the group consisting of: galectin-3 (LGALS3); HGF receptor (MET); single-chain type-1 glycoprotein (MIC2); or a combination thereof.

5. The acellular human amnion-derived composition of claim 1, further comprising one or more anti-proliferation biomolecules selected from the group consisting of: insulin-like growth factor-binding protein 4 (IGFBP4); ferritin (FTH1); or a combination thereof.

6. The acellular human amnion-derived composition of claim 1, wherein the composition comprises pentraxin 3 (PTX3).

7. The acellular human amnion-derived composition of claim 1, wherein the composition comprises HGF receptor (MET).

8. The acellular human amnion-derived composition of claim 1, wherein the composition comprises c-x-c motif chemokine 14 (CXCL14).

9. The acellular human amnion-derived composition of claim 1, wherein the composition comprises dickkopf WNT signaling pathway inhibitor 3 (DKK3).

10. The acellular human amnion-derived composition of claim 1, wherein the composition comprises low density lipoprotein receptor (LDLR).

11. The acellular human amnion-derived composition of claim 1, wherein the composition comprises transforming growth factor beta-induced protein ig-h3 (BIGH3).
